(19) United States
(12) Patent Application Publication
(10) Pub. No.: US 2009/0257976 A1
(43) Pub. Date: Oct. 15, 2009

(54) SINGLE VIAL FORMULATION FOR MEDICAL GRADE CYANOACRYLATE

(75) Inventors: Charles W. Kerber, La Mesa, CA (US); H. Clark Adams, San Diego, CA (US); Peter Friedman, La Jolla, CA (US)

Correspondence Address: KNOBBE MARTENS OLSON & BEAR LLP, 2040 MAIN STREET, FOURTEENTH FLOOR, IRVINE, CA, 92614, US

(73) Assignee: VALOR MEDICAL, INC., San Diego, CA (US)

(21) Appl. No.: 12/268,318

(22) Filed: Nov. 10, 2008

Related U.S. Application Data
(60) Provisional application No. 60/987,349, filed on Nov. 12, 2007.

(51) Int. Cl. A61K 31/785; C07C 255/08

(52) U.S. Cl. 424/78.35; 558/372

(57) ABSTRACT

Alkyl cyanoacrylate compositions and methods for making those compositions, utilizing high purity monomeric starting materials, formed into more viscous oligomers, and combined with a plasticizer and inhibitor to provide a single-container, storage stable medical cyanoacrylate.

### CROSS-REFERENCE TO RELATED APPLICATIONS

This application claims priority to U.S. Provisional Patent Application No. 60/987,349, filed on Nov. 12, 2007, entitled “Single Vial Formulation For Medical Grade Cyanoacrylate.”

### BACKGROUND OF THE INVENTION

1. Field of the Invention

This invention relates to storage stable cyanoacrylates that are useful for embolizing vascular aneurysms or otherwise occluding body spaces or lumens, utilizing alkyl cyanoacrylates with an alkyl chain length of four or more alkyl carbon atoms. It also relates to single vial formulations of cyanoacrylates useful in vivo and to methods for making cyanoacrylate monomers suitable for such single vial formulations.

2. Description of the Related Art

A cerebral aneurysm is a balloon-like swelling of the wall of a blood vessel in the brain. This weakening in the wall often leads to rupture, bleeding and death. Cerebral aneurysms are more common in people over 65, and they may be found in as high a 5% of the population. Smoking and hypertension appear to markedly increase the chance that one will develop a cerebral aneurysm. It is estimated that approximately 30,000 people in the United States are diagnosed each year with a cerebral aneurysm. However, there are an estimated 4.5 million individuals in the U.S. that have silent, undiagnosed cerebral aneurysm. This population is expected to grow with the aging of the population.

Aneurysms can be treated with direct (cranial) surgery or an endovascular approach. Direct surgery, under general anesthesia, is performed by opening the skull and identifying the neck of the aneurysm. This is the junction between the normal blood vessel and the weakened ballooned aneurysm. If possible, a clip is put across this area. This procedure involves cranial surgery, a lengthy procedure that requires several days hospitalization.

Endovascular surgery, also under general anesthesia, is performed by navigating a small tube or catheter into the aneurysm from the blood vessel in the leg artery under X-Ray guidance. Tiny platinum coils are used to fill the aneurysm. Patient selection is based on the individual patient and aneurysm anatomy. An endovascular coil placement procedure can take up to 3-5 hours and require multiple, 6-12, coils to be placed.

With the advent of the compositions and method disclosed herein, embolization with a cyanoacrylate composition represents a practical alternative to titanium coil placement, requiring less surgical time and producing a better result.

U.S. Pat. No. 6,037,366, which is incorporated herein in its entirety, discloses cyanoacrylate compositions which involve mixing two separate components immediately prior to administration.

Component I consists of a cyanoacrylate liquid monomer containing pure phosphoric acid (250 ppm) hydroquinone (100 ppm) and 4-methoxyphenol (1200 ppm). This component is stable and unchanging for over two years. Although stable upon long term storage the container in which component I is stored requires cleaning and preparation before such stability can be achieved. The preferred liquid monomer for use in component I is 2-hexyl cyanoacrylate.

Component II consists of pure powdered gold, a small amount of partial polymer of the same cyanoacrylate and plasticizer. The preferred plasticizer is ethyl myristate, but any liquid large chain fatty acid esters may work in this formulation. The patent discloses that the partial polymers of cyanoacrylate are unstable and change their structures and properties even in the solid state. Additionally the patent discloses that the change is exponential and the polymer must be used within a limited amount of time before deterioration occurs.

U.S. Pat. No. 6,476,069, which is incorporated herein in its entirety, discloses cyanoacrylate compositions that are prepared and maintained as a monomeric component and second component. The two components are mixed at the point of use.

The monomer component can be an alkyl cyanoacrylate and at least one inhibitor. Disclosed examples of the monomer component consist of cyanoacrylate monomer and at least one inhibitor. In one example the monomer component is comprised of 2-hexyl cyanoacrylate, hydroquinone, 4-methoxyphenol and phosphoric acid.

The second component can be a composition comprising, a opacificant material, such as gold, platinum, tantalum, titanium, tungsten and barium sulfate and the like, blended together with alkyl cyanoacrylate polymer material, and an esterified fatty acid, such as ethyl myristate. The monomer component and the second component are separately packaged and are mixed immediately prior to use as an embolic agent.

U.S. Pat. No. 6,476,070, which is incorporated herein in its entirety, discloses an invention known by the name of Neuracryl M, where Neuracryl M1 corresponds to the monomer component, and Neuracryl M2 corresponds to the second component comprising of the gold coated 2-hexyl acrylate. Neuracryl M is a two-part embolization agent consisting of a glass ampule of 1.25 ml Neuracryl M1 and a rubber-stoppered glass vial of Neuracryl M2 (a mixture of 2-hexyl cyanoacrylate, an esterified fatty acid, and gold particles. Prior to use, the contents of the Neuracryl M1 vial are injected into the vial containing Neuracryl M2, and the two are shaken together thoroughly until mixed. The gold particles and esterified fatty acid are used to retard polymerization and provide radiopacity. To avoid separation of the components or contamination, the two moieties were not mixed until immediately before use.

U.S. Pat. No. RE39, 150, which is incorporated herein in its entirety, discloses a composition that is a cyanoacrylate which involves mixing two separate containers of the material immediately prior to administration of the material. The composition may contain seven ingredients which are divided into two parts prior to mixture and use.

The embolic agents disclosed in the aforementioned patents require a two vial formulation. The contents of the two vials are combined to form the embolic agent immediately prior to clinical use. The embolic agents formed from the two vial formulations, must be used immediately. Alternatively, some have proposed compositions of fully polymerized materials (including cyanoacrylates) that are dissolved in a solvent, for embolic treatment. These solutions rely on post-injection dispersion of the solvent (e.g., DMSO) to precipitate the polymer at the site of use. Mixing at the point of use is typically required for around 30 minutes. The patient receives a significant dose of solvent (with risks attendant thereto) and the precipitation process is not sufficiently controllable, nor is the precipitated product satisfactory.

We have now developed compositions and methods relating to polymerizable cyanoacrylate agents, for vascular embolic and other related uses, that in at least some embodiments have one or more of the following desirable properties: appropriate cohesiveness, a robust rubbery casting, radiopacity, are well tolerated by the subject, and/or can be produced and packaged in a storage-stable single vial formulation.

### SUMMARY OF THE INVENTION

One embodiment disclosed herein includes a medical grade composition suitable for application to or in the human body, comprising a mixture of (a) a polymerizable alkyl cyanoacrylate monomer or oligomer; (b) at least one polymerization inhibitor; (c) a contrast agent; and (d) a plasticizer, wherein the composition is sealed in a single container and is stable for more than one month at room temperature, and is adapted to polymerize in vivo.

One embodiment disclosed herein includes a method of preparing an alkyl cyanoacrylate monomer of formula (I)

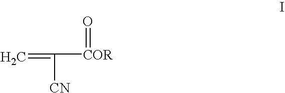

where R is alkyl of 4 to 10 carbon atoms, comprising:
(a) reacting formaldehyde with a compound of formula (1-A)

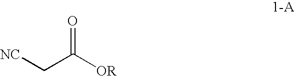

in the presence of a catalyst to provide a partial polymer-(alkyl cyanoacrylate), wherein R is as defined above in connection with Formula I;
(b) adding a first inhibitor selected from the group consisting of 4-methoxyphenol, 2,6-di-tert-butyl-4-methylphenol, SO₂ and any combinations thereof to the partial polymer-alkyl cyanoacrylate;
(c) cracking the partial polymer-alkyl cyanoacrylate to provide a cracked alkyl cyanoacrylate in a container containing a second inhibitor selected from the group consisting of 4-methoxyphenol, 2,6-di-tert-butyl-4-methylphenol, SO₂ and any combinations thereof;
(d) distilling the cracked alkyl cyanoacrylate of (c) to provide a alkyl cyanoacrylate monomer distillate in a container containing a third inhibitor selected from the group consisting of 4-methoxyphenol, 2,6-di-tert-butyl-4-methylphenol, SO₂ and any combinations thereof; and
(e) removing the third inhibitor from the alkyl cyanoacrylate monomer distillate.

One embodiment disclosed herein includes a method of preparing a medical grade alkyl cyanoacrylate composition in a single container comprising, (a) treating an alkyl cyanoacrylate monomer photochemically to provide an alkyl cyanoacrylate oligomer having a viscosity of from about 5 to about 1000 centipoise; and (b) combining the alkyl cyanoacrylate oligomer with a plasticizer solution comprising a plasticizer and an inhibitor, to provide an alkyl cyanoacrylate oligomer plasticizer mixture.

One embodiment disclosed herein includes a method of providing a single container alkyl cyanoacrylate formulation comprising,
(a) providing an alkyl cyanoacrylate oligomer that has been formed by irradiating a of monomer of formula (I) to partially polymerize the monomer;

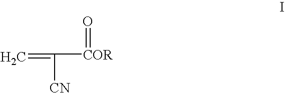

where R is alkyl of 4 to 10 carbon atoms,
wherein the alkyl cyanoacrylate monomer of formula (I) has a viscosity of from about 3 centipoise to about 5 centipoise; and
the alkyl cyanoacrylate oligomer has a viscosity of from about 10 centipoise to about 1000 centipoise;
(b) combining the alkyl cyanoacrylate oligomer with a plasticizer and an inhibitor to provide an alkyl cyanoacrylate oligomer plasticizer mixture; and
(c) placing the resulting alkyl cyanoacrylate oligomer plasticizer mixture in a single container, such that the resulting single container alkyl cyanoacrylate formulation is stable for more than one month at room temperature, and is adapted to polymerize in vivo.

One embodiment disclosed herein includes a composition comprising:
(a) an alkyl cyanoacrylate oligomer;
(b) at least one inhibitor;
(c) an opacificant agent; and
(d) a plasticizer
wherein the alkyl cyanoacrylate oligomer has been prepared from alkyl cyanoacrylate monomer;
wherein said composition is in a single container and is stable for more than one month, and
when said composition contacts an anionic environment it polymerizes to form an aggregate structure.

One embodiment disclosed herein includes a composition comprising:
(a) an alkyl cyanoacrylate oligomer, wherein 30% to 50% of the composition by weight is said alkyl cyanoacrylate oligomer, wherein the alkyl cyanoacrylate oligomer has a viscosity of from about 15 centipoise to about 500 centipoise
(b) a plasticizer mixture, wherein 10% to 30% of said composition by weight is said plasticizer mixture
(c) an opacificant agent, wherein 30% to 50% of the composition by weight is said opacificant agent
wherein said composition is in a single container and is stable for more than one month.

One embodiment disclosed herein includes a method of preparing an embolic agent comprising
(a) mixing an alkyl cyanoacrylate oligomer with a plasticizer solution to provide an alkyl cyanoacrylate oligomer plasticizer solution
(b) combining the alkyl cyanoacrylate oligomer plasticizer solution with an opacificant agent in a single container to provide a pre-sterilization mixture
(c) storing the pre-sterilization mixture under an inert atmosphere
(d) heating the single container containing the pre-sterilization mixture to a temperature sufficient to sterilize the pre-sterilization mixture
wherein the alkyl cyanoacrylate oligomer has a viscosity of from about 15 centipoise to about 500 centipoise; and
the embolic agent is stable for more than one month.

One embodiment disclosed herein includes a formulation for body space remodeling, comprising;
an alkyl cyanoacrylate in an amount up to about 50 weight percent;
a plasticizer mixture in an amount up to about 30 weight percent, wherein the plasticizer mixture consists of an acyl trialkyl citrate, 4-methoxyphenol, 2,6-di-tert-butyl-4-methylphenol, sulfur dioxide, and combinations thereof; and
a opacificant agent in an amount up to 50 weight percent wherein the contrast agent is selected from the group consisting of gold, platinum, tantalum, titanium, tungsten and barium sulfate; and
the formulation is chemically and physically stable upon storage at room temperature for at least 30 days in a single vial.

One embodiment disclosed herein includes a kit for embolizing a body lumen, comprising a formulation for body space remodeling in a single container and a catheter or syringe configured to introduce the embolotherapy product into the body lumen, wherein the kit includes written instructions or information.

### DETAILED DESCRIPTION OF PREFERRED EMBODIMENTS

Although alkyl cyanoacrylates have been proposed in the past for embolization of aneurysms and for filling other spaces in the body, those early research efforts have not led to the availability of such products and methods for general clinical treatment. This is due, in part, to formulation deficiencies, including the need to mix two components on site prior to use, or the need to inject a cyanoacrylate polymer dissolved in significant quantities of solvent. We have discovered, contrary to the expectation and general understanding in the art, that formulations of medically-useful, polymerizable alkyl cyanoacrylates can be prepared and stored in a single vial, for longer than 1 month. Some embodiments of the present invention provide a composition comprising a single vial formulation of an oligomer of at least one alkyl cyanoacrylate monomer, at least one inhibitor, an opacificant agent and a plasticizer.

In some embodiments, the oligomer component can be one or more alkyl cyanoacrylate oligomers, and at least one inhibitor. In a preferred embodiment the oligomer component can be a n-hexyl cyanoacrylate. In a preferred embodiment the composition can include multiple (e.g., three) inhibitors, for example, the inhibitors can be hydroquinone, 4-methoxyphenol and phosphoric acid. In a typical embodiment the inhibitors can be 2,6-di-tert-butyl-4-methylphenol, 4-methoxyphenol and sulfur dioxide (SO₂).

Some embodiments can comprise a method for purifying alkyl cyanoacrylate monomer to its crystalline form. In some embodiments, the method of purifying an alkyl cyanoacrylate can provide an alkyl cyanoacrylate monomer with a purity of about 95% or better. In a preferred embodiment, the alkyl cyanoacrylate monomer has a purity of about 97% or better. In a more preferred embodiment, the alkyl cyanoacrylate monomer has a purity of about 98% or better. In an especially preferred embodiment, the alkyl cyanoacrylate monomer is at least 99% pure.

Some embodiments comprise a substantially pure alkyl cyanoacrylate monomer or oligomer. For example, the alkyl cyanoacrylate monomer can be methyl cyanoacrylate, n-butyl cyanoacrylate, isobutyl cyanoacrylate, n-hexyl cyanoacrylate, 2-hexyl cyanoacrylate or 2-octyl cyanoacrylate, purified to about 95% purity or better. In a preferred embodiment, methyl cyanoacrylate, n-butyl cyanoacrylate, isobutyl cyanoacrylate, n-hexyl cyanoacrylate, 2-hexyl cyanoacrylate or 2-octyl cyanoacrylate can be purified to about 97% purity or better. In a more preferred embodiment methyl cyanoacrylate, n-butyl cyanoacrylate, isobutyl cyanoacrylate, n-hexyl cyanoacrylate, 2-hexyl cyanoacrylate or 2-octyl cyanoacrylate can be purified to about 98% purity or better. In a most preferred embodiment methyl cyanoacrylate, n-butyl cyanoacrylate, isobutyl cyanoacrylate, n-hexyl cyanoacrylate, 2-hexyl cyanoacrylate or 2-octyl cyanoacrylate can be purified to about 99% purity or better. In some aspects the alkyl cyanoacrylate monomer can be isolated in its crystalline form.

The stability of formulations made from alkyl cyanoacrylate monomers can be related to the purity of the monomer that is used. These properties can include but are not limited to rate of polymerization, and stability of the monomer during storage. An advantage of substantially pure alkyl cyanoacrylates can be that compositions incorporating substantially pure alkyl cyanoacrylates can require smaller amounts of additives, e.g., inhibitors, stabilizers and the like, to obtain a desired result that would otherwise have required greater amounts of the same additive. A benefit of this advantage can be in cost savings from being able to use less material. Another benefit can be that the composition will quantitatively have lower amounts of additives. This can be a desirable outcome for any composition that is subject to regulatory approval by the U.S. Food and Drug Administration, or like agency, prior to marketing. Of significant importance is the ability of the product to form a shelf-stable formulation.

It is believed that the inability of the prior art to provide single vial formulations of alkyl cyanoacrylates, particularly formulations that include alkyl cyanoacrylate monomer (or in the case of some present embodiments, oligomer) having 4, 5, 6, or more carbons in the alkyl chain, was due to alkyl cyanoacrylate monomer stability. Thus, some aspects of the invention provide suitably pure monomer, and single vial formulations including or made from an oligomer of that monomer.

Some embodiments include compositions that have partially polymerized cyanoacrylate oligomer therein. We have discovered that the use of ultraviolet light to partially polymerize the alkyl cyanoacrylate monomers results in an advantageous product profile that is particularly suited to single vial formulations with extended shelf life.

Some embodiments can provide alkyl cyanoacrylate monomers whose rates of polymerization can be predicted, and the un-reacted monomer (“pre-polymer”) compositions are more stable. One of ordinary skill in the art can select the monomer with the appropriate polymerization properties for a desired use, or to formulate monomer compositions having desired polymerization properties. Previously, alkyl cyanoacrylates (especially those with alkyl chains of 4, 5, 6, or more carbons) have not been available in substantially pure form because they can be difficult to purify using conventional chemical methodology. Moreover, most of these methodologies involve conditions that cause the alkyl cyanoacrylate to degrade or to spontaneously polymerize. Heretofore, such substantially pure alkyl cyanoacrylate monomers with desired chain lengths (such as 4, 5, and especially 6 carbon alkyl groups) were not heretofore generally available.

Another embodiment of the present invention can provide a method for filling, occluding, partially filling or partially occluding an unfilled volume or space in vivo. Use of the compositions disclosed herein as embolic agents for filling vascular aneurysms is particularly preferred.

### DEFINITIONS

As used herein the term “alkyl cyanoacrylate” refers to an adhesive compound or mixture of compounds based on cyanoacrylate monomers of formula I:

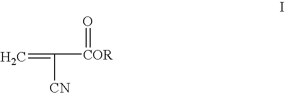

where R is selected from the group consisting of alkyl of one to sixteen carbon atoms. Partial polymers (i.e., oligomers) of such cyanoacrylates are also encompassed within this definition. Preferred R alkyl group are from 4 to 8 carbon atoms and include, by way of example, methyl, ethyl, n-butyl, isobutyl, pentyl, n-hexyl, 2-hexyl, n-heptyl, 2-heptyl, n-octyl and 2-octyl. More preferably, R is n-hexyl, 2-hexyl, isobutyl, 2-heptyl and 2-octyl and most preferably, R is n-hexyl.

As used herein the term “partial polymers” or “oligomers” indicates a polymer consisting of only a few monomer units such as a dimer, trimer, tetramer, etc., or their mixtures. The mean number of monomers in the partial polymer can typically be up to about ten, or if polymerization is allowed to continue, the number of monomer units can advantageously be between 10 and 100. The “partial polymers” or “oligomers” can be further polymerized to form polymers.

As used herein the term “polymer” indicates a substance composed of molecules with large molecular mass composed of repeating structural units, or monomers. The “polymer” is defined as generally being at the limit of polymerization in the particular formulation in which the polymerization took place, so that polymerization is substantially complete. This is in contrast to a “partial polymer” or “oligomer,” which can be prepared by partially polymerizing a composition, such that substantial further polymerization is possible.

As used herein the term “alkyl” refers to a carbon chain of one to sixteen carbon atoms, where the carbon atoms can be linear or branched.

As used herein the term “lower-alkyl” refers to a carbon chain of one to eight carbon atoms, where the carbon atoms can be linear or branched. Examples of lower-alkyl moieties include but are not limited to methyl, ethyl, n-butyl, isobutyl, pentyl, n-hexyl, 2-hexyl, n-heptyl, 2-heptyl, n-octyl and 2-octyl.

As used herein the term “branched alkyl” refers to a carbon chain of one to sixteen carbon atoms where the carbon chain contains at least one secondary or tertiary substituted carbon atom.

As used herein the term “branched lower-alkyl” refers to a carbon chain of one to eight carbon atoms where the carbon chain contains at least one secondary or tertiary substituted carbon atom, for example, 2-hexyl, isobutyl, 2-heptyl and 2-octyl.

As used herein the term “biocompatible plasticizer” refers to any material which is soluble or dispersible in alkyl cyanoacrylate, which increases the flexibility of the resulting polymer coating on the skin surface, and which is compatible with the skin as measured by the lack of skin irritation. Suitable plasticizers are well known in the art and include those disclosed in U.S. Pat. Nos. 2,784,127 and 4,444,933 the disclosures of both of which are incorporated herein by reference in their entirety. Specific plasticizers include, by way of example only, butyl benzyl phthalate, dibutyl phthalate, diethyl phthalate, dimethyl phthalate, dioctylphthalate, trialkyl acylcitrates, benzoate esters of di- and poly-hydroxy branched aliphatic compounds, tri(p-cresyl) phosphate, and the like. The plasticizer employed advantageously is selected to avoid skin irritation. Preferred plasticizers for use in this invention are acyl trialkyl citrates independently having from 1 to 10 carbon atoms in each alkyl group. For example, acyl trialkyl acylcitrates include but are not limited to trimethyl O-acetylcitrate, triethyl O-acetylcitrate, tri-n-propyl O-acetylcitrate, tri-n-butyl O-acetylcitrate, tri-n-pentyl O-acetylcitrate, tri-n-hexyl O-acetylcitrate, tri-methyl O-propionylcitrate, tri-ethyl O-propionylcitrate, tri-n-propyl O-propionylcitrate, tri-n-butyl O-propionylcitrate, tri-n-pentyl O-propionylcitrate, tri-n-hexyl O-propionylcitrate, tri-methyl O-butyrylcitrate, tri-ethyl O-butyrylcitrate, tri-n-propyl O-butyrylcitrate, tri-n-butyl O-butyrylcitrate, tri-n-pentyl O-butyrylcitrate, tri-n-hexyl O-butyrylcitrate, and the like. A typical plasticizer is tri-n-butyl O-acetylcitrate.

As used herein the term “anionic environment” refers to an environment that has free anions, such as OH—. Anions in water and other aqueous media, such as blood, can catalyze the polymerization of cyanoacrylates.

As used herein the terms “adhesion” or “adhesive” means the characteristic or tendency of a material to be attracted to the surface of a second material. Adhesion occurs as the result of interactions between two materials. Depending on the characteristics of the second material relative to the first material, adhesion may or may not occur.

As used herein the term “cohesion” or “cohesive” means the characteristic or tendency of a material to stick together to itself. For example, this characteristic is demonstrated by a material or composition remaining intact as a single mass when introduced into a stationary fluid, or a fluid stream in motion, such as, blood. Lack of cohesive integrity results in the composition breaking up into multiple smaller subunits.

As used herein, the term “microparticulate” means a small particle of 200 mesh (0.075 mm) or smaller, preferably 400 mesh or smaller.

As used herein the term “alkyl esterified fatty acid” means a fatty acid derivatized to form an ester functional group with a alkyl moiety, such as ethyl myristate. These compounds are formed with an alkyl moiety, such as methyl, ethyl, propyl, butyl, pentyl, hexyl, heptyl, and octyl; and carboxylic acids with alkyl side chains ranging from 1 carbon, i.e., acetic acid, through to and including 17 carbons atoms in length, such as, proprionic, butyric, isobutyric, valeric, isovaleric, pivalic, lauric, myristic, palmitic and stearic acids.

As used herein the term “opacificant agent” is compound or composition which selectively absorbs or deflects radiation making the material visible under x-ray or other types of imaging, such as MRI and ultrasound. Typically, x-ray contrast agents include iodinated oils and brominated oils, as well as commercially available compositions, such as Pantopaque, Lipiodol and Ethiodol. These commercially available compositions acts as opacificant agents, and also dilute the amount of liquid monomer, thereby slowing the rate of polymerization. In addition, certain metals, such as, gold, platinum, tantalum, titanium, tungsten and barium sulfate and the like, have properties enabling them to act as opacificant agents. It is preferred that these opacificant agents, particularly metals, have a smooth, regular, preferably spherical morphology. This can improve the viscosity and flow characteristics of the final product.

As used herein the term “polymerization” refers to the chemical process where a monomer unit or partial polymer unit chemically reacts with other monomer units or partial polymer units to form larger aggregates that contain the monomer unit or partial polymer unit.

As used herein the term “full polymerization” refers to the chemical process where a monomer unit, partial polymer unit, or oligomer chemically reacts to form a polymer that does not undergo significant additional chain-lengthening polymerization.

As used herein the term “polymer” refers to a substance composed of molecules with large molecular mass composed of repeating structural units, or monomers, connected by covalent chemical bonds.

As used herein the term “partial polymer” refers to a substance composed of repeating structural units, or monomers, connected by covalent chemical bonds whereby the substance can undergo further chain-lengthening polymerization to form a polymer.

As used herein the term “a space” refers to an unfilled volume or cavity in a patient.

As used herein the term “stability” refers to the ability of a compound or formulation to resist degradation or polymerization after preparation but prior to use.

As used herein the term “inhibitor” refers to an agent which stabilizes an alkyl cyanoacrylate monomer or alkyl cyanoacrylate single vial formulation by inhibiting polymerization. Within the context of the current invention, this term refers to an intrinsic agent or agents that stabilize and inhibit polymerization by at least one mechanism. By altering the amounts of one or more inhibitors, the rate of polymerization can be controlled. Inhibitors have different modes of activity, for example, hydroquinone acts primarily to inhibit high energy free radicals; 4-methoxyphenol acts primarily to inhibit low energy free radicals; and phosphoric acid influences the rate of anionic polymerization.

In some embodiments, the composition can be formed from alkyl cyanoacrylate monomeric and/or oligomeric units, such as, methyl, n-butyl, isobutyl, n-hexyl and 2-hexyl cyanoacrylate with at least one inhibitor, such as hydroquinone, 4-methoxyphenol and phosphoric acid. In preferred embodiments, the composition can form into a polymer when it comes in contact with an anionic environment, such as blood or tissue.

Compositions of the present invention can advantageously possess several or all of the following properties.

1) The composition can be prepared and maintained in a single vial combination of a polymerizable component and opacificant for an appreciable length of time.
2) The composition has the ability to reliably and predictably change from a liquid state to a solid state in vivo.
3) The composition has sufficiently low viscosity to enable administration via syringes, catheters, cannulas, tubes, or other like devices.
4) The composition has cohesive characteristics such that when the composition in administered into an aqueous fluid environment, such as blood, the composition forms a single polymerized structure (including heterogeneous structures).
5) The rate of heat released during polymerization of the composition is low enough such that the heat does not adversely affect surrounding tissues that may be heat sensitive.
6) The composition and its biodegradation products are sufficiently non-histotoxic and non-cytotoxic that its presence is well tolerated in the body.

Cyanoacrylates can generate heat as they change from monomeric to oligomeric or polymeric form. The amount and rate of heat released, if excessive, can have a detrimental effect on the living tissue proximate to the vessel. Control of the amount and rate at which heat is released during polymerization can be important in some applications of the technology.

### Preparation of the Monomer Component

The monomer component of the present invention is prepared by forming the desired precursor ester from the corresponding alkyl alcohol and cyanoacetic acid resulting in the desired alkyl cyanoacetate as depicted in Scheme A. The starting materials for this reaction are commercially available, for example from Sigma-Aldrich Chemical Company, VWR, Fisher, Lancaster or Fluka Chemical Company, or can be prepared following procedures known to those of ordinary skill in the art.

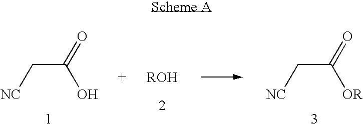

The compound of Formula 2 can be any alkyl alcohol, where R is from one to sixteen carbons, including but not limited to alcohols based on alkyl groups, such as, methyl, ethyl, propyl, butyl, pentyl, hexyl, heptyl, heptyl, octyl, nonyl, deca, undeca, dodeca, trideca, tetradeca, pentadeca and hexadeca, where the preceding moieties are linear (e.g., n-propyl, n-butyl, n-pentyl, or n-hexyl) or variously branched, such as sec-butyl, iso-butyl, tert-butyl, iso-propyl, 2-butyl, 2-pentyl, 2-hexyl, 2-heptyl, 2-octyl and the like. Particularly advantageous alcohols are those disclosed in U.S. Pat. No. 3,728,375 entitled “Cyanoacrylate Adhesive Compositions”, which is hereby incorporated in its entirety. Especially preferred alcohols can be selected from the group consisting of methyl, n-butyl, iso-butyl, n-hexyl and 2-hexyl alcohols.

In some embodiments, about 1 molar equivalent of the compounds of Formula 1 and Formula 2 are combined in a solvent such as toluene at about 100 ml/molar equivalents. To this mixture can be added a catalytic amount (about 1.0×10⁻⁴ molar equivalents) of p-toluene sulfonic acid. The mixture can be stirred and heated to reflux. The preparation can ideally yield the desired alkyl cyanoacetate at a purity level of about 95%. The experimental conditions can be readily modified by one of ordinary skill in the art without deviating from the present invention. Aspects such as solvent selection, reaction time, temperature and choice of reagents are well within the skill of one of ordinary skill in the art. If necessary, the material can be further purified using multiple distillations and purification techniques and procedures known to those of ordinary skill in the art, such as water extraction, vacuum distillation, column chromatography, and the like. It is preferred that the alkyl cyanoacetate be substantially free from impurities. In one embodiment, the cyanoacetate has a purity of from about 95% to about 100%.

Preparation of Alkyl Cyanoacrylates

The desired alkyl cyanoacrylate monomer component of the present invention can be synthesized from the alkyl cyanoacetate by reacting the it in a Knoevengel type reaction as depicted in Scheme B.

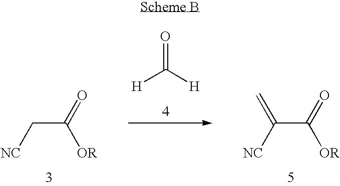

In some embodiments, about 1 molar equivalent of formaldehyde (Formula 4), which is prepared from paraformaldehyde, and piperidine (at about 0.33 ml/molar equivalents) can be combined in a solvent, such as methanol (at about 166 ml/molar equivalents). To this mixture can be added about 1 molar equivalents of alkyl cyanoacetate (Formula 3) in a dropwise manner. The reaction mixture can be refluxed with stirring, yielding alkyl cyanoacrylate. The alkyl cyanoacrylate can be converted to monomer form by cracking and distillation. The reaction mixture can be further processed with about 0.2 to 0.7 molar equivalents, preferably about 0.2 to 0.6 molar equivalents of phosphorous pentoxide, yielding the desired purified alkyl cyanoacrylate. It is desirable to carefully perform the purification steps to prevent the compound of Formula (5) from polymerizing. To this end the system can be treated with trace amounts of sulfur dioxide, and receiver flasks can be treated with 4-methoxyphenol. After initial cracking and distillation, the desired alkyl cyanoacrylate monomer can be further purified using multiple distillations or other purification techniques known to those of ordinary skill in the art, such as vacuum distillation, spinning band column, and the like.

In one preferred embodiment, the following technique can be used for making purified alkyl cyanoacrylate of Formula I, where R is alkyl of 4-10 carbon atoms, preferably 5-10 or 5-8 carbon atoms, and most preferably 6 carbon atoms:

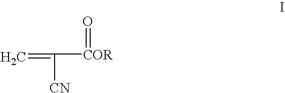

The synthesis is designed to produce alkyl cyanoacrylate of high purity that can be used to make single container (e.g., single vial) formulations of polymerizable alkyl cyanoacrylate for medical use. Care is taken to avoid contamination, polymerization, and degradation of the product by maintaining the reaction mixture and products under an appropriately-nonreactive atmosphere or vacuum, and through careful laboratory techniques.

One suitable synthetic scheme includes some or all of the following steps:
(a) combining paraformaldehyde prills, catalytic amine and a first solvent (e.g., methanol) in a reaction vessel. For example, the catalytic amine could be a secondary amine, such as, piperidine or diethyl amine;
(b) heating and stirring the contents of the reaction vessel to produce high quality formaldehyde (preferably at a temperature between about 65 and 80 C);
(c) reducing the heat applied to the vessel (preferably to about 55 C);
(d) adding a cyanoacetate compound of formula (1-A):

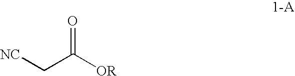

to the vessel, wherein R is as defined above in connection with Formula I;
(e) increasing the heat applied to the vessel to react the formaldehyde and the cyanoacetate to form alkyl cyanoacrylate (preferably at about 72° C. and to about 78° C.);
(f) removing solvent (e.g., methanol) from the alkyl cyanoacrylate by distilling off between 75% to 95% of the liquid volume contained in the reaction vessel; and then allowing the vessel and contents to cool;
(g) after removal of the first solvent, adding a second solvent (e.g., toluene) to the flask to form a mixture with the contents of the flask;
(h) distilling off from about 85% to about 100% of the solvent volume contained in the flask, including azeotropic distillation of remaining first solvent and catalytic amine; and then allowing the reaction vessel and contents to cool;
(i) collecting alkyl cyanoacrylate monomer of formula (I) as a distillate in a receiving flask containing inhibitor, such as 4-methoxyphenol, 2,6-di-tert-butyl-4-methylphenol, hydroquinone or any combinations thereof;
(j) placing the reaction vessel under vacuum, wherein the vacuum is preferably from about 5 mm Hg to about 0.1 mm Hg, more preferably from about 2 mm Hg to about 0.5 mm Hg;
(k) heating the reaction vessel to remove residual solvents by vacuum distillation, preferably at a temperature below about 150° C.;
(l) discontinuing heating the reaction vessel, wherein the reaction vessel and contents are allowed to cool;
(m) breaking the vacuum with a non-reactive gas, such as argon, nitrogen, sulfur dioxide, and combinations thereof;
(n) blanketing the reaction apparatus with SO₂.
(o) placing the reaction vessel under vacuum, wherein the vacuum is preferably from about 5 mm Hg to about 0.1 mm Hg, more preferably from about 2 to about 0.5 mm Hg;
(p) heating the reaction vessel to a sufficiently high temperature to vaporize the alkyl cyanoacrylate, wherein the temperature preferably does not exceed 200° C., and is preferably between about 170° C. to about 190° C.;
(q) collecting alkyl cyanoacrylate monomer of formula (I) as a distillate in a receiving flask containing inhibitor, such as 4-methoxyphenol, 2,6-di-tert-butyl-4-methylphenol, hydroquinone or any combinations thereof;
(r) discontinuing heating;
(s) breaking the vacuum with a nonreactive gas, such as argon and nitrogen, and combinations thereof;
(t) blanketing the reaction apparatus with SO₂;
(u) following collection of the distilled alkyl cyanoacrylate, removing the stabilizer, e.g., with inhibitor-remover such as Aldrich 311332, to obtain an alkyl cyanoacrylate monomer of formula (I), with a viscosity of from about 3 centipoise to about 8 centipoise, typically about 4 centipoise; and
(v) removing SO₂ by bubbling or sparging a nonreactive gas, such as argon, nitrogen, or combinations thereof through the alkyl cyanoacrylate.

In one embodiment, an alkyl cyanoacrylate monomer can be treated to form an alkyl cyanoacrylate oligomer with viscosity greater than the alkyl cyanoacrylate monomer. For example, an n-hexyl cyanoacrylate monomer can be treated to form an n-hexyl cyanoacrylate oligomer. Preferred oligomers result from photochemical treatment of an alkyl cyanoacrylate monomer, to produce a mixture that includes partially polymerized cyanoacrylate and that has a higher viscosity than the alkyl cyanoacrylate monomer from which it is formed. For example, the viscosity of the alkyl cyanoacrylate oligomer can advantageously be from about 10 centipoise to about 1000 centipoise and the viscosity of the alkyl cyanoacrylate monomer, from which the alkyl cyanoacrylate oligomer is formed, can be from about 2 centipoise to about 5 centipoise as measured by a rheometer. Typically, the oligomer will have a viscosity of from about 10 centipoise to about 50 centipoise.

In a typical embodiment, an n-hexyl cyanoacrylate monomer, with a viscosity of about 4 centipoise, is treated to form an n-hexyl cyanoacrylate oligomer, with a viscosity of about 15 centipoise to about 50 centipoise. In an exemplary embodiment, an n-hexyl cyanoacrylate monomer, with a viscosity of about 4 centipoise, can be treated to form an n-hexyl cyanoacrylate oligomer, with a viscosity of about 25 centipoise to about 30 centipoise. In some embodiments, the alkyl cyanoacrylate monomer can be treated photochemically to provide the alkyl cyanoacrylate oligomer. In a typical embodiment, UV radiation can be used for the photochemical treatment. In an exemplary embodiment, UV radiation can provide very controlled polymerization of alkyl cyanoacrylate monomer to form alkyl cyanoacrylate oligomer capable of further polymerization. Some techniques for polymerizing cyanoacrylate monomers using irradiation are disclosed in U.S. Patent Publication 20050197421, incorporated herein by reference in its entirety. In typical embodiment, an n-hexyl cyanoacrylate monomer, with a viscosity of about 4 centipoise, can be treated with UV radiation to form an n-hexyl cyanoacrylate oligomer, with a viscosity of about 15 centipoise to about 50 centipoise. In another exemplary embodiment, an n-hexyl cyanoacrylate monomer, with a viscosity of about 4 centipoise, can be treated with UV radiation to form an n-hexyl cyanoacrylate oligomer, with a viscosity of about 25 centipoise to about 30 centipoise. In some embodiments, the UV radiation source can be a Mercury vapor lamp. In a typical embodiment, a 550 watt Mercury vapor lamp in an Ace Glass Incorporated photochemical reactor is used for the photochemical treatment.

Formulation

In some embodiments, the alkyl cyanoacrylate component of the present invention is combined with least one inhibitor. For example, typical inhibitors appropriate for alkyl cyanoacrylates can be, for example, hydroquinone, 4-methoxyphenol, pure phosphoric acid, 2,6-di-tert-butyl-4-methylphenol, sulfur dioxide (SO₂), alkyl carboxylic acids, and the like. Mixtures of inhibitors are also contemplated. In a typical embodiment, two or more of 4-methoxyphenol, 2,6-di-tert-butyl-4-methylphenol, and sulfur dioxide (SO₂), can be used, individually or in combination.

Different inhibitors have different physical characteristics and thereby function to alter the final properties of the composition. For example, hydroquinone can be used as an inhibitor for high energy free radicals; 4-methoxyphenol can be used as an inhibitor for low energy free radicals and phosphoric acid can act to control or inhibit anionic polymerization and the rate of such polymerization.

The quantity of inhibitors used can be measured in terms of parts per million of alkyl cyanoacrylate. For example, for n-hexyl cyanoacrylate, hydroquinone can be in the range of about 50 to about 500 parts per million (PPM), 4-methoxyphenol can be in the range of about 50 to about 500 PPM, 2,6-di-tert-butyl-4-methylphenol can be in the range of about 50 to about 500 PPM, SO₂ can be in the range of about 25 to about 300 PPM and phosphoric acid can be in the range of about 125 to about 375 PPM. In a typical embodiment, hydroquinone can be in the range of about 100 to about 350 PPM, 4-methoxyphenol can be in the range of about 100 to about 350 PPM, 2,6-di-tert-butyl-4-methylphenol can be in the range of about 100 to about 350 PPM, SO₂ can be in the range of about 50 to about 250 PPM and phosphoric acid can be in the range of about 185 to about 300 PPM. In a more typical embodiment, 4-methoxyphenol can be in the range of about 200 to about 300 PPM, 2,6-di-tert-butyl-4-methylphenol can be in the range of about 200 to about 300 PPM, and SO₂ can be in the range of about 75 to about 125 PPM.

In some embodiments, the composition can include a opacificant material, such as gold, platinum, tantalum, titanium, tungsten and barium sulfate and the like; an alkyl cyanoacrylate oligomer, and a plasticizer. Suitable opacificants or contrast agents are further disclosed, for example, in US Patent Publication No. 20050287216, incorporated herein by reference in its entirety. In some embodiments, the plasticizer can be butyl benzyl phthalate, dibutyl phthalate, diethyl phthalate, dimethyl phthalate, dioctylphthalate, trialkyl acylcitrates, benzoate esters of di- and poly-hydroxy branched aliphatic compounds, tri(p-cresyl) phosphate, combinations thereof and the like. In an exemplary embodiment, plasticizers can be acyl trialkyl citrates independently having from 1 to 10 carbon atoms in each alkyl group. For example, acyl trialkyl acylcitrates can be trimethyl O-acetylcitrate, triethyl O-acetylcitrate, tri-n-propyl O-acetylcitrate, tri-n-butyl O-acetylcitrate, tri-n-pentyl O-acetylcitrate, tri-n-hexyl O-acetylcitrate, tri-methyl O-propionylcitrate, tri-ethyl O-propionylcitrate, tri-n-propyl O-propionylcitrate, tri-n-butyl O-propionylcitrate, tri-n-pentyl O-propionylcitrate, tri-n-hexyl O-propionylcitrate, tri-methyl O-butyrylcitrate, tri-ethyl O-butyrylcitrate, tri-n-propyl O-butyrylcitrate, tri-n-butyl O-butyrylcitrate, tri-n-pentyl O-butyrylcitrate, tri-n-hexyl O-butyrylcitrate, and the like. In a typical embodiment, the plasticizer can tri-n-butyl O-acetylcitrate.

In some embodiments, the opacificant is a microparticle or nanoparticle of a liquid or solid contrast agent suspended in an alkyl cyanoacrylate. In a typical embodiment, the opacificant can be a solid contrast agent, such as, gold, platinum, tantalum, titanium, tungsten and barium sulfate and the like. In a more typical embodiment, solid contrast agent can be gold suspended in an alkyl cyanoacrylate oligomer. For example, the gold can be suspended in n-hexyl cyanoacrylate oligomer. Factors that influence the amount of opacificant can include the amount of opacificant necessary for fluoroscopic detection.

In some embodiments, the sealed storage containers can be heated sterilized as disclosed in Disinfection, Sterilization, and Preservation, Seymour S Block ed., Lippincott Williams & Wilkins, 2000 incorporated herein in its entirety. For example, the sealed storage containers can be heated sterilized form about 120° C. to about 190° C. In a typical embodiment, the sealed storage containers can be heated sterilized at 180° C. for from about 3 to about 15 min. In a more typical embodiment, the sealed storage containers can be heated sterilized at 180° C. for from about 4 to about 6 min.

Prior art compositions containing alkyl cyanoacrylate polymer typically use a solvent or alkyl cyanoalkylate monomer to solubilize the alkyl cyanoacrylate polymer, and at least some of the compositions contemplated herein do not have substantial amounts of solvent, particularly when alkyl cyanoacrylate oligomer, alkyl cyanoacrylate monomer, stabilizer, and contrast agent are excluded from the definition of solvent.

In some embodiments, the composition includes alkyl cyanoacrylate oligomer, inhibitor, and opacificant free of solvent. In an embodiment, the amount of solvent can be less than about 10% w/w, typically the amount of solvent can be less than about 5% w/w, more typically the amount of solvent can be less than about 3%, 1%, or 0.5% w/w of the composition.

In one embodiment, the composition is also largely or substantially free from cyanoacrylate monomer (except such monomer as may remain incidentally when a monomer is partially polymerized to create an oligomer). Alternatively, the amount of monomer may be sufficiently limited that it would be insufficient to solubilize cyanoacrylate polymer used as a viscosity modifying agent.

In some embodiments, the alkyl cyanoacrylate formulations can be useful for filling, occluding, partially filling or partially occluding an unfilled volume or space in a mass (“a space”). In particular, the composition is useful for filling vascular aneurysms. The composition has the property of polymerizing when it comes in contact with an aqueous environment, such as when it contacts blood or when it is deployed in situ in another most cavity or space in the body.

The materials disclosed herein can often be prepared and maintained as a monomeric component or single vial formulation until needed. They have the ability to reliably and predictably change from a liquid state to a solid state, which is important for its administration through catheters, cannulas, syringes, and the like.

In some embodiments, the composition can be administered into an aqueous fluid environment, such as blood, where the composition forms a single aggregate structure. In other words, it is sufficiently cohesive before, during and after polymerization in vivo that crumbling, disintegration, and separation are minimized or eliminated. The rate of heat released during polymerization of the present invention can be low enough that the heat does not adversely effect surrounding tissues that may be heat sensitive.

In some embodiments, the alkyl cyanoacrylate polymer can be sufficiently non-histotoxic and non-cytotoxic so that its presence can be well tolerated in the body. In some embodiments, the composition of the present invention can be useful for filling, occluding, partially filling or partially occluding an unfilled volume or space in a mass.

Administration

In some embodiments, the alkyl cyanoacrylate formulations can be utilized in various medical procedures, including, without limitation, treatment of cerebral aneurysms, arterio-venous malformations, treatment of uterine fibroid, treatment of abdominal aortic aneurysms, and endoleaks resulting from vascular stent placement.

In one embodiment, for example, in treating a cerebral aneurysm, a microcatheter is advanced to the location of the aneurysm under direct fluoroscopic observation. If desired, vascular flow is inhibited during the procedure with an occlusion balloon. A single vial formulation of medical grade alkyl cyanoacrylate formulation is transferred through the catheter and into the aneurysm, until the aneurysm is filled. Careful attention is given to maintaining monolithic integrity of the implanted material, so that no particles or pieces separate from the implanted mass. The cyanoacrylate polymerizes as it is placed due to contact with blood. The polymerizing cyanoacrylate is shaped during and after placement with a balloon or other tool to create a smooth surface for blood flow past the aneurysm site, with an effort to provide laminar blood flow. This greatly reduces the risk of thrombus formation and stroke resulting therefrom. If the aneurysm is at a branch in a vessel, the cyanoacrylate can be shaped to create a wedge-shaped flow divider, with the point of the wedge extending in the opposite direction of blood flow, directing, and dividing blood flow smoothly into each downstream vessel.

In a typical embodiment, the alkyl portion of the cyanoacrylate has an alkyl chain length greater than 4, more typically 5-10 or 5-7, and most typically 6. N-hexyl cyanoacrylate is particularly preferred, due to a confluence of desirable morphological, viscosity, and biocompatible characteristics when combined with the other components of the formulation. These include a cohesive structure, such that no pieces separate from the body of the implanted material; a viscosity allowing injection through a microcatheter while remaining in one mass upon injection; low toxicity; good in vivo tolerability; and workability during the procedure to allow shaping for optimal blood flow.

Some embodiments disclosed herein include medical grade compositions suitable for application to or in the human body, comprising a mixture of:
(a) a polymerizable alkyl cyanoacrylate monomer or oligomer;
(b) at least one polymerization inhibitor;
(c) a contrast agent; and
(d) a plasticizer
wherein said composition can be sealed in a single container and can be stable for more than one month at room temperature, and can be adapted to polymerize in vivo.

In some embodiments, the alkyl cyanoacrylate can be an oligomer. For example, the oligomer can be 2-hexyl cyanoacrylate, n-hexyl cyanoacrylate pentyl cyanoacrylate, heptyl cyanoacrylate, octyl cyanoacrylate and the like. In an exemplary embodiment, the oligomer can be n-hexyl cyanoacrylate oligomer.

In some embodiments, the medical grade composition can include a plasticizer. For example, the plasticizer can be butyl benzyl phthalate, dibutyl phthalate, diethyl phthalate, dimethyl phthalate, dioctylphthalate, trialkyl acylcitrates, benzoate esters of di- and poly-hydroxy branched aliphatic compounds, tri(p-cresyl) phosphate, and the like. In some embodiments, the plasticizer can be an acyl trialkyl citrates independently having from 1 to 10 carbon atoms in each alkyl group. For example, acyl trialkyl acylcitrates include but are not limited to trimethyl O-acetylcitrate, triethyl O-acetylcitrate, tri-n-propyl O-acetylcitrate, tri-n-butyl O-acetylcitrate, tri-n-pentyl O-acetylcitrate, tri-n-hexyl O-acetylcitrate, tri-methyl O-propionylcitrate, tri-ethyl O-propionylcitrate, tri-n-propyl O-propionylcitrate, tri-n-butyl O-propionylcitrate, tri-n-pentyl O-propionylcitrate, tri-n-hexyl O-propionylcitrate, tri-methyl O-butyrylcitrate, tri-ethyl O-butyrylcitrate, tri-n-propyl O-butyrylcitrate, tri-n-butyl O-butyrylcitrate, tri-n-pentyl O-butyrylcitrate, tri-n-hexyl O-butyrylcitrate, and the like. In a typical embodiment, the plasticizer can be tri-n-butyl O-acetylcitrate.

In some embodiments, the medical grade composition can include an inhibitor, For example, the inhibitor can be 4-methoxyphenol, 2,6-di-tert-butyl-4-methylphenol, hydroquinone, phosphoric acid, sulfur dioxide (SO₂), any combination thereof and the like. In a typical embodiment, the inhibitor can be 4-methoxyphenol, 2,6-di-tert-butyl-4-methylphenol, sulfur dioxide and any combinations.

In some embodiments, the medical grade composition can include a contrast agent. For example, the contrast agent can be gold, platinum, tantalum, titanium, tungsten and barium sulfate, any combinations, and the like. In a typical embodiment, the contrast agent can be gold.

In some embodiments, the medical grade composition can include both alkyl cyanoacrylate monomer and alkyl cyanoacrylate oligomer. In some embodiments, the alkyl cyanoacrylate oligomer can have a viscosity from about 5 centipoise to about 1000 centipoise. In a typical embodiment, the alkyl cyanoacrylate oligomer can have a viscosity from about 10 centipoise to about 100 centipoise. In a more typical embodiment, the alkyl cyanoacrylate oligomer can have a viscosity from about 15 centipoise to about 35 centipoise.

In some embodiments, the medical grade composition can be substantially free from viscosity-modifying amounts of an alkyl cyanoacrylate polymer.

In some embodiments, the medical grade composition can be in a single container that is substantially opaque to ultraviolet light and/or to visible light. This does not necessarily require that the container be completely opaque; instead, in addition to complete opacity, a partially opaque vial (e.g., brown glass) can be used. Alternatively, in some embodiments, the medical grade composition can be in a single container that is transparent or translucent to visible light.

Some embodiments disclosed herein include methods of preparing an alkyl cyanoacrylate monomer of formula (I)

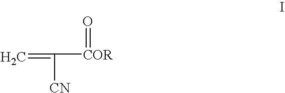

where R is alkyl of 4 to 10 carbon atoms, comprising:
(a) reacting formaldehyde with a compound of formula (1-A)

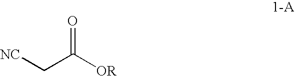

in the presence of a catalyst to provide a partial polymer-alkyl cyanoacrylate, wherein R is as defined above in connection with Formula I;
(b) adding a first inhibitor selected from the group consisting of 4-methoxyphenol, 2,6-di-tert-butyl-4-methylphenol, SO₂ and any combinations thereof to the partial polymer-alkyl cyanoacrylate;
(c) cracking the partial polymer-alkyl cyanoacrylate to provide a cracked alkyl cyanoacrylate in a container containing a second inhibitor selected from the group consisting of 4-methoxyphenol, 2,6-di-tert-butyl-4-methylphenol, SO₂ and any combinations thereof;
(d) distilling the cracked alkyl cyanoacrylate of (c) to provide an alkyl cyanoacrylate monomer distillate in a container containing a third inhibitor selected from the group consisting of 4-methoxyphenol, 2,6-di-tert-butyl-4-methylphenol, SO₂ and any combinations thereof;
(f) removing the third inhibitor from the alkyl cyanoacrylate monomer distillate.

In some embodiments, the purity of the alkyl cyanoacrylate monomer of formula (I) can be from 95% to 100%. In a typical embodiment, the purity of the alkyl cyanoacrylate monomer of formula (I) can be from 98% to 100%. In a more typical embodiment, the purity of the alkyl cyanoacrylate monomer of formula (I) can be from 99% to 100%.

Some embodiments disclosed herein include methods of preparing a medical grade alkyl cyanoacrylate composition in a single container comprising,
(a) treating an alkyl cyanoacrylate monomer photochemically to provide an alkyl cyanoacrylate oligomer that can have a viscosity of from about 5 to about 1000 centipoise;
(b) mixing the alkyl cyanoacrylate oligomer with a plasticizer solution where the plasticizer solution comprises a plasticizer and an inhibitor to provide an alkyl cyanoacrylate oligomer plasticizer mixture.

In some embodiments, the plasticizer of the plasticizer solution in the medical grade composition can comprise butyl benzyl phthalate, dibutyl phthalate, diethyl phthalate, dimethyl phthalate, dioctylphthalate, trialkyl acylcitrates, benzoate esters of di- and poly-hydroxy branched aliphatic compounds, tri(p-cresyl) phosphate, any combinations thereof and the like. In some embodiments, the plasticizer can be an acyl trialkyl citrate independently having from 1 to 10 carbon atoms in each alkyl group. For example, an acyl trialkyl acylcitrate can include trimethyl O-acetylcitrate, triethyl O-acetylcitrate, tri-n-propyl O-acetylcitrate, tri-n-butyl O-acetylcitrate, tri-n-pentyl O-acetylcitrate, tri-n-hexyl O-acetylcitrate, tri-methyl O-propionylcitrate, tri-ethyl O-propionylcitrate, tri-n-propyl O-propionylcitrate, tri-n-butyl O-propionylcitrate, tri-n-pentyl O-propionylcitrate, tri-n-hexyl O-propionylcitrate, tri-methyl O-butyrylcitrate, tri-ethyl O-butyrylcitrate, tri-n-propyl O-butyrylcitrate, tri-n-butyl O-butyrylcitrate, tri-n-pentyl O-butyrylcitrate, tri-n-hexyl O-butyrylcitrate, any combinations thereof and the like. In a typical embodiment, the plasticizer can be tri-n-butyl O-acetylcitrate.

In some embodiments, of the plasticizer solution in the medical grade composition can be 4-methoxyphenol, 2,6-di-tert-butyl-4-methylphenol, hydroquinone, phosphoric acid, sulfur dioxide (SO₂), any combinations, and the like.

In some embodiments, the medical grade composition can include combining an opacificant agent with the alkyl cyanoacrylate oligomer plasticizer mixture. In some embodiments, the opacificant agent can be gold, platinum, tantalum, titanium, tungsten and barium sulfate, any combinations, and the like. In a typical embodiment, the opacificant agent can be gold.

In some embodiments, the single container medical grade composition can be stored in single container that is opaque to visible light. Alternatively, in some embodiments, the medical grade composition can be stored in single container that is transparent or translucent to visible light.

Some embodiments disclosed herein include methods of providing a single container alkyl cyanoacrylate formulation comprising,
(a) providing an alkyl cyanoacrylate monomer of formula (I)

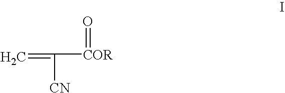

where R is alkyl of 4 to 10 carbon atoms,
that can be irradiated with ultraviolet radiation to form an alkyl cyanoacrylate oligomer,
wherein the alkyl cyanoacrylate monomer of formula (I) can have a viscosity of from about 3 centipoise to about 5 centipoise; and
the alkyl cyanoacrylate oligomer can have a viscosity of from about 10 centipoise to about 1000 centipoise
(b) combining the alkyl cyanoacrylate oligomer with a plasticizer and an inhibitor to provide an alkyl cyanoacrylate oligomer plasticizer mixture
(c) placing the resulting alkyl cyanoacrylate oligomer plasticizer mixture in a single container, such that the resulting single container alkyl cyanoacrylate formulation can be stable for more than one month at room temperature, and can be adapted to polymerize in vivo.

In some embodiments, the plasticizer of the single container alkyl cyanoacrylate formulation can be an acyl trialkyl citrate. In some embodiments, the inhibitor of the single container alkyl cyanoacrylate formulation can be 4-methoxyphenol, 2,6-di-tert-butyl-4-methylphenol, hydroquinone, phosphoric acid, sulfur dioxide (SO₂), and any combinations.

In some embodiments, the single container alkyl cyanoacrylate formulation can include an opacificant agent. For example, the opacificant agent can be selected from the group consisting of gold, platinum, tantalum, titanium, tungsten, an iodine compound, barium sulfate and the like.

In some embodiments, the single container of the single container alkyl cyanoacrylate formulation can be opaque to visible light. Alternatively, in some embodiments, the single container of the single container alkyl cyanoacrylate formulation can be transparent or translucent to visible light.

Some embodiments disclosed herein include compositions comprising:
(a) an alkyl cyanoacrylate oligomer;
(b) at least one inhibitor;
(c) an opacificant agent; and
(d) a plasticizer
wherein the alkyl cyanoacrylate oligomer can be prepared from alkyl cyanoacrylate monomer;
wherein said composition can be in a single container and can be stable for more than one month, and
when said composition contacts an anionic environment it can polymerize to form an aggregate structure.

In some embodiments, in the composition comprising, an alkyl cyanoacrylate oligomer, at least one inhibitor, an opacificant agent and a plasticizer, the alkyl cyanoacrylate oligomer can be n-hexyl cyanoacrylate oligomer. In a typical embodiment, the n-hexyl cyanoacrylate oligomer can have a viscosity of 5 to 1000 centipoise. In a more typical embodiment, n-hexyl cyanoacrylate oligomer can have a viscosity of 15 to 100 centipoise. In a most typical embodiment, n-hexyl cyanoacrylate oligomer can have a viscosity of 20 to 35 centipoise.

In some embodiments, in the composition comprising, an alkyl cyanoacrylate oligomer, at least one inhibitor, an opacificant agent and a plasticizer, the inhibitor can be 4-methoxyphenol, 2,6-di-tertbutyl-4-methylphenol sulfur dioxide (SO₂), hydroquinone, phosphoric acid, any combinations and the like.

In some embodiments, in the composition comprising, an alkyl cyanoacrylate oligomer, at least one inhibitor, an opacificant agent and a plasticizer, the opacificant agent can be selected from the group consisting of gold, platinum, tantalum, titanium, tungsten, barium sulfate and the like. In a typical embodiment, the opacificant agent can be gold.

In some embodiments, in the composition comprising, an alkyl cyanoacrylate oligomer, at least one inhibitor, an opacificant agent and a plasticizer, the single container can be opaque to visible light. Alternatively, in some embodiments, the single container can be transparent or translucent to visible light.

In some embodiments, in the composition comprising, an alkyl cyanoacrylate oligomer, at least one inhibitor, an opacificant agent and a plasticizer, the single container can include sulfur dioxide. In a typical embodiment, the amount of sulfur dioxide in the composition can be from 5 ppm to 500 ppm. In a more typical embodiment, the amount of sulfur dioxide in the composition can be from 10 ppm to 100 ppm.

Some embodiments disclosed herein include methods of preparing an embolic agent comprising,
(a) mixing an alkyl cyanoacrylate oligomer with a plasticizer solution to provide an alkyl cyanoacrylate oligomer plasticizer solution
(b) combining the alkyl cyanoacrylate oligomer plasticizer solution with an opacificant agent in a single container to provide a pre-sterilization mixture
(c) storing the pre-sterilization mixture under an inert atmosphere
(d) heating the single container containing the pre-sterilization mixture to a temperature sufficient to sterilize the pre-sterilization mixture
wherein the alkyl cyanoacrylate oligomer can have a viscosity of from about 15 centipoise to about 500 centipoise; and
the embolic agent can be stable for more than one month.

In some embodiments, the plasticizer solution of the embolic agent can be selected from an acyl trialkyl citrate, one or more inhibitors, and any combinations thereof. In a typical embodiment, the plasticizer solution can be selected from tri-n-butyl O-acetylcitrate p-methoxyphenol, 2,6-di-tert-butyl-4-methylphenol, sulfur dioxide, and any combinations.

In some embodiments, the opacificant agent of the embolic agent can be gold, platinum, tantalum, titanium, tungsten, barium sulfate and any combinations thereof. In a typical embodiment, the opacificant agent can be gold.

In some embodiments, the pre-sterilization mixture can be sterilized. For example, the pre-sterilization mixture can be sterilized by irradiation or heat. In a typical embodiment the pre-sterilization mixture can be sterilized by heat. For example, in an exemplary embodiment, the temperature sufficient to sterilize the pre-sterilization mixture is from about 150° C. to about 200° C.

In some embodiments, a single container containing an embolic agent can be opaque to visible light. Alternatively, in some embodiments, the single container can be transparent or translucent to visible light.

Some embodiments disclosed herein include formulations for body space remodeling, comprising;

an alkyl cyanoacrylate in an amount up to about 50 weight percent;

a plasticizer mixture in an amount up to about 30 weight percent, wherein the plasticizer mixture consists of an acyl trialkyl citrate, 4-methoxyphenol, 2,6-di-tert-butyl-4-methylphenol, sulfur dioxide, and combinations thereof; and

a opacificant agent in an amount up to 50 weight percent wherein the contrast agent can be selected from the group consisting of gold, platinum, tantalum, titanium, tungsten and barium sulfate; and

the formulation can be chemically and physically stable upon storage at room temperature for at least 30 days in a single vial. In preferred embodiments, the formulation is stable for at least about 45 days, preferably 2, 3, 4, 5, or 6 months, and most preferably one year, 18 months, or two years, at room temperature.

In some embodiments, an alkyl cyanoacrylate of the formulation for body space remodeling can be a n-hexyl cyanoacrylate. In a typical embodiment, the n-hexyl cyanoacrylate can be a n-hexyl cyanoacrylate oligomer.

In some embodiments, the acyl trialkyl citrate of the formulation for body space remodeling can be tri-n-butyl O-acetylcitrate.

In some embodiments, the opacificant agent of the formulation for body space remodeling can be gold.

Some embodiments disclosed herein include kits for embolizing a body lumen, comprising a formulation for body space remodeling in a single container and a catheter or syringe configured to introduce the embolotherapy product into the body lumen, wherein the kit includes written instructions or information.

Some embodiments can include methods of preparing an alkyl cyanoacrylate monomer of formula (I) where R can be alkyl of 4 to 10 carbon atoms

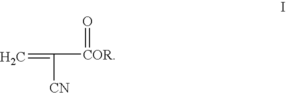

In a typical embodiment, an alkyl cyanoacrylate monomer of formula (I) can be prepared by reacting formaldehyde with a compound of formula (1-A)

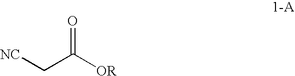

in the presence of a secondary amine in a solvent to form an alkyl cyanoacrylate, wherein R is as defined above in connection with Formula I. For example, the secondary amine can be piperidine, diethylamine and the like. In some embodiments, the solvent can then be removed by distillation to provide an alkyl cyanoacrylate residue. For example, the alkyl cyanoacrylate residue can be a partial polymer, polymer and the like.

In some embodiments, a solvent can be added to the alkyl cyanoacrylate residue to form an alkyl cyanoacrylate solvent mixture. Typically, the solvent can be a solvent that produces and azeotrope with residual solvents that may remain after the first solvent removal. For example, the solvent can be a solvent such as toluene, benzene and the like. In some embodiments, the solvent can be removed. For example, the solvent can be azeotropically removed by distillation to provide a crude alkyl cyanoacrylate, such as a crude partial polymer alkyl cyanoacrylate, crude polymer alkyl cyanoacrylate, combinations thereof and the like.

In some embodiments, polyphosphoric acid and inhibitor can be added to the alkyl cyanoacrylate. In a typical embodiment, the inhibitor can be hydroquinone, 4-methoxyphenol, 2,6-di-tert-butyl-4-methylphenol, SO₂, any combinations thereof and the like. Additionally, in some embodiments, any residual solvent can be removed under vacuum. For example, residual solvent can be removed by vacuum distillation.

In some embodiments, the crude alkyl cyanoacrylate can be heated to provide an alkyl cyanoacrylate monomer. For example the crude alkyl cyanoacrylate can be depolymerized and a gaseous alkyl cyanoacrylate monomer can be collected into a container containing an inhibitor. In a typical embodiment, crude alkyl cyanoacrylate can be depolymerized by cracking. For example, the crude alkyl cyanoacrylate can be heated to from about 150° C. to about 210° C. under a vacuum of from about 5 mmHg to about 0.1 mmHg. The gaseous alkyl cyanoacrylate monomer can be collected into a vessel containing and inhibitor by condensation. In one embodiment, the vacuum does not exceed 200° C. and the vacuum is from 5 mmHg to about 1 mmHg. In some embodiments, the vacuum can be broken with an inert gas, for example argon or nitrogen, and the system blanketed with SO₂.

In some embodiments, the alkyl cyanoacrylate monomer can be further purified by distillation. For example vacuum distillation. In a typical embodiment, the alkyl cyanoacrylate monomer can be distilled under vacuum and collected in a container containing an inhibitor. For example the inhibitor can be hydroquinone, 4-methoxyphenol, 2,6-di-tert-butyl-4-methylphenol, SO₂, any combinations thereof and the like. In an exemplary embodiment, the vacuum can be from 5 mmHg to about 0.1 mmHg. In a most typical embodiment, the vacuum can be from 5 mmHg to about 1 mmHg and the inhibitors can be 4-methoxyphenol, 2,6-di-tert-butyl-4-methylphenol, SO₂, any combinations thereof and the like. Further, in some embodiments, the vacuum can be broken with an inert gas, such as argon, nitrogen and the like. In some embodiments, the system can then be blanketed with SO₂ to provide a pure alkyl cyanoacrylate monomer inhibitor mixture.

In some embodiments, the inhibitor(s) can be removed by an inhibitor remover and the SO₂ can be removed by bubbling an inert gas through the a pure alkyl cyanoacrylate monomer solution to provide the pure alkyl cyanoacrylate monomer free of inhibitors.

### EXAMPLES

The following examples are given to enable those of ordinary skill in the art to more clearly understand and to practice the present invention. The examples should not be considered as limiting the scope of the invention, but merely as illustrative and representative thereof.

### Example 1

### Preparation of Stabilized n-Hexyl Cyanoacrylate

Step (a) Initial Reaction

Formaldehyde frills (290 g, 9.7 moles) were added to a 3000 mL 3-necked reactor, equipped with a Dean-Stark distillation apparatus, followed by 650 mL methanol and finally 4.8 mL piperidine. The reaction mixture was stirred using an overhead stirrer and heating was initiated. The mixture was heated to between 65° C. and 80° C. and maintained in this range for 45 minutes, during which time the solution became “milky”. The temperature was reduced to ˜55° C. and n-hexyl cyanoacetate (1600 g, 8.8 moles) was slowly added. During the addition of the n-hexyl cyanoacetate, the temperature was maintained between 68° C. and 75° C. The reaction mixture color became yellowish toward the completion of the addition. An additional 100 mL methanol was used to rinse residual n-hexyl cyanoacetate into the reaction mixture via the addition funnel.

The reaction was heated to reflux and approximately 610 ml methanol was removed via Dean-Stark distillation over ˜1 hour (during which the temperature of the reaction increased from 72° C. to 78° C.) at which time the n-hexyl cyanoacrylate was formed. Subsequently, 630 ml toluene was added via an addition funnel. The mixture containing the n-hexyl cyanoacrylate was heated to remove the residual methanol and piperidine via azeotropic distillation, which occurred from 84° C. to 115° C. (uncorrected temperature). When the temperature rose to 115° C. the distillation was discontinued. The system was allowed to cool to room temperature.

Step (b) Cracking process

The reaction apparatus was reassembled to replace the Dean-Stark distillation apparatus setup with a Vigreux distillation column. A chilled condenser with a receiver flask was attached to the distillation column. The system was set up so a vacuum could be applied as necessary. To the reaction vessel was added 50 mg polyphosphoric acid and 0.8 g 4-methoxyphenol and then the system was sealed.

The receiver flask was cooled with liquid nitrogen and then the mixture was stirred and the system placed under vacuum (5 mm Hg to 1 mm Hg). The vacuum was regulated by bleeding in argon. The reaction vessel was maintained below 150° C. and a liquid fraction containing all the added toluene was collected by distillation. The vacuum was broken using argon and then the system was blanketed with SO₂ for 3 seconds. The receiver flask containing toluene was replaced with a pre-weighed collection vessel containing 4-methoxyphenol (10 mg/100 mL vessel size, e.g. a 1 L vessel contains 100 mg of 4-methoxyphenol). The apparatus was placed under vacuum (5 mmHg to 1 mm Hg), and the reaction vessel was heated to from about 170° C. to about 190° C. (not to exceed 200° C.) to initiate cracking of the polymer, the n-hexyl cyanoacrylate monomer distills at 80° C. to 95° C. at the above stated vacuum. A forerun of 50 mL to 100 mL of n-hexyl cyanoacrylate was collected and discarded, breaking the vacuum with argon and blanketing the system with SO₂ for 3 seconds. The receiver flask containing the forerun was replaced with a pre-weighed collection vessel containing 4-methoxyphenol (10 mg/100 mL vessel size, e.g. a 1 L vessel contains 100 mg of 4-methoxyphenol). The apparatus was placed under vacuum (5 mmHg to 1 mm Hg), and the reaction vessel was heated to from about 170° C. to about 190° C. (not to exceed 200° C.) to initiate cracking of the polymer, the monomer distills at 80° C. to 95° C. at the above stated vacuum. When no further pale yellow n-hexyl cyanoacrylate monomer was collected, the heating was stopped, the vacuum was broken with argon and the system blanketed with SO₂ for 3 seconds. The rate of collection of the monomer is approximately 1 L per day, including the steps of exchanging collection vessels. Note that in the preceding process, care was taken to maintain a non-reactive atmosphere over the reaction mixture and resulting product, thus avoiding unwanted polymerization and degradation reactions. This, in turn, enhances the quality and purity of the end product, such that it is stable in a single vial formulation.

Step (c) Distilling process

A vacuum distillation apparatus was configured with a 2 L flask (3-neck round bottom flask), magnetic stirrer, and a Vigreux column. The distillation apparatus was placed under argon and then the pale yellow n-hexyl cyanoacrylate distillate from the cracking step was added to the distillation flask. The apparatus was maintained under argon and blanketed with SO₂ for 3 seconds and stirring of the liquid in the distillation flask was initiated. The receiving flask was cooled with liquid nitrogen and then the distillation apparatus was placed under vacuum (5 mmHg to 1 mm Hg). The pale yellow n-hexyl cyanoacrylate was gradually heated with stirring until distillation initiated. Distillate was collected at a rate of one drop per minute. After ˜50 ml of forerun was collected the vacuum was broken with argon, followed by blanketing with SO₂. The forerun was discarded and a second receiving flask containing 4-methoxyphenol ((10 mg/100 mL vessel size) was placed to receive the distillate. Several fractions of distillate were collected so that the final 100 mL of distillate could be discarded. During each flask exchange the vacuum was broken with argon and the system was blanketed with SO₂. Pure n-hexyl cyanoacrylate was collected containing 4-methoxyphenol and SO₂ for use in the next step.

### Example 2

### Photochemical Viscosity Adjustment of n-Hexyl Cyanoacrylate Monomer

The purified n-hexyl cyanoacrylate monomer from Example 1, containing 4-methoxyphenol, was treated with Aldrich HQ & MEHQ inhibitor remover, Sigma-Aldrich, Inc., St. Louis, Mo., USA (2005-2006 Catalog #306320), to remove the p-methoxyphenol, followed by bubbling argon through the n-hexyl cyanoacrylate monomer to remove SO₂. The viscosity of the purified n-hexyl cyanoacrylate, free of 4-methoxyphenol and SO₂, was 4 centipoise.

The purified n-hexyl cyanoacrylate (500 g) was then introduced into an Ace glass photochemical reactor equipped medium pressure quartz mercury vapor lamp. The n-hexyl cyanoacrylate was irradiated until the liquid had a viscosity of about 20 to about 35 centipoise. The resulting oligomer material is referred to as Component A. This viscosity modification tailors the end product for use in the vasculature of a patient, with sufficiently high viscosity to allow the injected composition to remain where it is placed, in one intact mass, while at the same having a sufficiently low viscosity to allow it to be injected through a microcatheter.

### Example 3

### Preparation of Plasticizer Component

A stock solution of tri-n-butyl O-acetylcitrate containing 4-methoxyphenol and 2,6-di-tert-butyl-4-methylphenol was prepared as follows. To tri-n-butyl O-acetylcitrate (500 grams, 1.24 mol) under argon was added 4-methoxyphenol (750 PPM) and 2,6-di-tert-butyl-4-methylphenol (750 PPM). The mixture was stirred until homogeneous. Sulfur dioxide (SO₂, 600 PPM) was bubbled through the tri-n-butyl O-acetylcitrate solution containing 4-methoxyphenol and 2,6-di-tert-butyl-4-methylphenol. The resulting material is referred to as Component B.

### Example 4

### Component C: Formulation of Component A with Component B

The UV treated n-hexyl cyanoacrylate (Component A, 500 g) was combined with Component B (250 g) at room temperature and mixed until homogeneous. The viscosity of the resulting product was from about 20 to about 35 centipoise. The above combination of Component A and Component B affords Component C.

### Example 5

### Preparation of Single Vial Formulation

Component C (1.5 mL) is added to a 5 mL vial containing fine mesh gold (0.9 g,) and the vial is placed under argon. The vial is then sealed and heat sterilized. The single vial formulation is stable for over 1 year.

### Example 6

### Preparation of 2-Hexyl Cyanoacrylate

This prospective procedure is based on procedures developed employed for preparing n-hexyl cyanoacrylate, as is taught in the preceding example.

Equip a 5 liter three-necked flask with a reflux condenser, Dean-Stark trap, an addition funnel and a mechanical stirrer with a glass paddle in a 5 liter heating mantle. To the flask is added the following components, prills of paraformaldehyde (136 g, 4.5 moles), methanol (300 mL) and pyridine (2.2 mL). The reaction mixture is stirred and heated to between 65° C. and 80° C. for 45 min. The heating is cooled to 55° C. and 2-hexyl cyanoacetate (736 g, 4.1 moles) is added drop wise via an addition funnel. The reaction is exothermic and the rate of addition should be adjusted to keep the reaction mixture temperature between 68° C. and 75° C. An additional 46 mL of methanol is used to rinse the addition funnel. Collect the methanol distilled from the reaction flask through the Dean-Stark trap. Measure the amount recovered. Continue the distillation until 80% or more of the original volume of methanol is recovered over a one hour period of time. Subsequently, toluene (290 mL) is added via the addition funnel. The mixture is heated to remove the residual methanol and piperidine via azeotropic distillation, the distillation occurs from 84° C. to 115° C. (uncorrected temperature). When the temperature reaches 115° C. the distillation is discontinued. The system is allowed to cool to room temperature before reaction apparatus is reassembled for the next step

The reaction apparatus is reassembled to replace the Dean-Stark distillation apparatus setup with a Vigreux distillation column to which a chilled condenser was attached and a receiver flask. The system is set up so a vacuum can be applied as necessary. To the reaction vessel is added polyphosphoric acid (23 mg) and 4-methoxyphenol (0.37 g) and then the system is sealed.

The receiver flask was cooled with liquid nitrogen and then the mixture was stirred and the system placed under vacuum (5 mmHg to 1 mm Hg). the vacuum is regulated by bleeding in argon. The reaction vessel is maintained below 150° C. and a liquid fraction containing remaining toluene is collected. The applied vacuum is isolated from the system and the vacuum is broken with argon. Subsequently, the system is blanketed under SO₂ for 3 seconds.

The vacuum is broken using argon and then the system was placed under SO₂ for 3 seconds. The collection vessel containing distillate is replaced with a pre-weighed collection vessel containing 4-methoxyphenol (10 mg/100 mL vessel size, e.g. a 1 L vessel contains 100 mg of 4-methoxy phenol). The reaction apparatus is placed under vacuum (0.1-0.5 mm Hg), and the reaction vessel is heated to from about 170° C. to about 190° C. (not to exceed 200° C.) to initiate cracking of the polymer. A forerun of 50 mL to 100 mL of 2-hexyl cyanoacrylate was collected and discarded, breaking the vacuum with argon and blanketing the system with SO₂ for 3 seconds. The receiver flask containing the forerun was replaced with a pre-weighed collection vessel containing 4-methoxyphenol (10 mg/100 mL vessel size, e.g. a 1 L vessel contains 100 mg of 4-methoxyphenol). The apparatus was placed under vacuum (5 mmHg to 1 mm Hg), and the reaction vessel was heated to from about 170° C. to about 190° C. (not to exceed 200° C.) to initiate cracking of the polymer, the monomer distills at 80° C. to 95° C. at the above stated vacuum. The collection vessel containing 2-hexyl cyanoacrylate monomer is replaced with another empty pre-weighed collection vessel containing 4-methoxyphenol (10 mg/100 mL vessel size) and the above process is repeated until the majority of the 2-hexyl cyanoacrylate monomer is collected (blanket with sulfur dioxide at each flask exchange). The rate of collection of the monomer is 1 L per day, including the steps of exchanging collection vessels.

### Example 7

### Preparation of n-Pentyl Cyanoacrylate

This prospective procedure is based on procedures developed employed for preparing n-hexyl cyanoacrylate, as is taught in the preceding examples.

Equip a 10 liter three-necked flask with a reflux condenser, Dean-Stark trap, an addition funnel and a mechanical stirrer. To the flask is added the following components, prills of paraformaldehyde (272 g, 9 moles), methanol (600 mL) and pyridine (4.4 mL). The reaction mixture is stirred and heated to between 65° C. and 80° C. for 45 min. The heating is removed and the mixture cools to ˜55° C. and then n-pentyl cyanoacetate (1372 g, 8.2 moles) is added drop wise via an addition funnel. The reaction is exothermic and the rate of addition should be adjusted to keep the reaction mixture temperature between 68° C. and 75° C. An additional 92 mL of methanol is used to rinse the addition funnel. The methanol is distilled from the reaction flask through the Dean-Stark trap and collected. The distillation is continued until 80% or more of the original volume of methanol is recovered over a one hour period of time. Subsequently, toluene (580 mL) was added via the addition funnel. The mixture is heated to remove the residual methanol and piperidine via azeotropic distillation, the distillation occurs from 84° C. to 115° C. (uncorrected temperature). When the temperature reaches 115° C. the distillation is discontinued. The system is allowed to cool to room temperature before reaction apparatus is reassembled for the next step

The reaction apparatus is reassembled to replace the Dean-Stark distillation apparatus setup with a Vigreux distillation column to which a chilled condenser was attached and a receiver flask. The system is set up so a vacuum can be applied as necessary. To the reaction vessel is added polyphosphoric acid (46 mg) and 4-methoxyphenol (0.74 g) and then the system is sealed.

The receiver flask is cooled with liquid nitrogen and then the mixture is stirred and the system is placed under vacuum (5 mmHg to 1 mm Hg). the vacuum is regulated by bleeding in argon. The reaction vessel is maintained below 150° C. and a liquid fraction containing remaining toluene is collected. The applied vacuum is isolated from the system and the vacuum is broken with argon. Subsequently, the system is blanketed under SO₂ for 3 seconds.

The vacuum is broken using argon and then the system was placed under SO₂ for 3 seconds. The collection vessel containing distillate is replaced with a pre-weighed collection vessel containing 4-methoxyphenol (10 mg/100 mL vessel size, e.g. a 1 L vessel contains 100 mg of 4-methoxy phenol). The reaction apparatus is placed under vacuum (0.1-0.5 mm Hg), and the reaction vessel is heated to from about 170° C. to about 190° C. (not to exceed 200° C.) to initiate cracking of the polymer. A forerun of 50 mL to 100 mL of n-pentyl cyanoacrylate was collected and discarded, breaking the vacuum with argon and blanketing the system with SO₂ for 3 seconds. The receiver flask containing the forerun was replaced with a pre-weighed collection vessel containing 4-methoxyphenol (10 mg/100 mL vessel size, e.g. a 1 L vessel contains 100 mg of 4-methoxyphenol). The apparatus was placed under vacuum (5 mmHg to 1 mm Hg), and the reaction vessel was heated to from about 170° C. to about 190° C. (not to exceed 200° C.) to initiate cracking of the polymer, the monomer distills at 80° C. to 95° C. at the above stated vacuum. The collection vessel containing n-pentyl cyanoacrylate monomer is replaced with another empty pre-weighed collection vessel containing 4-methoxyphenol (10 mg/100 mL vessel size) and the above process is repeated until the majority of the n-Pentyl cyanoacrylate monomer is collected (blanket with sulfur dioxide at each flask exchange). The rate of collection of the monomer is 1 L per day, including the steps of exchanging collection vessels.

What is claimed is:

1. A medical grade composition suitable for application to or in the human body, comprising a mixture of:
(a) a polymerizable alkyl cyanoacrylate monomer or oligomer;
(b) at least one polymerization inhibitor;
(c) a contrast agent; and
(d) a plasticizer,
wherein said composition is sealed in a single container and is stable for more than one month at room temperature, and is adapted to polymerize in vivo.

2. The composition of claim 1, wherein the alkyl cyanoacrylate is an oligomer and is selected from the group consisting of 2-hexyl cyanoacrylate, n-hexyl cyanoacrylate, pentyl cyanoacrylate, heptyl cyanoacrylate, and octyl cyanoacrylate.

3. The composition of claim 1, wherein the alkyl cyanoacrylate is an oligomer of n-hexyl cyanoacrylate.

4. The composition of claim 1, wherein the inhibitor is selected from the group consisting of 4-methoxyphenol, 2,6-di-tert-butyl-4-methylphenol, hydroquinone, phosphoric acid, sulfur dioxide (SO₂), and any combination thereof.

5. The composition of claim 4, wherein the inhibitor is 4-methoxyphenol, 2,6-di-tert-butyl-4-methylphenol, and sulfur dioxide.

6. The composition of claim 1, wherein the plasticizer is tri-n-butyl O-acetylcitrate.

7. The composition of claim 1, wherein the contrast agent is selected from the group consisting of gold, platinum, tantalum, titanium, tungsten and barium sulfate, and any combinations.

8. The composition of claim 7, wherein the contrast agent is gold.

9. The composition of claim 1, containing both alkyl cyanoacrylate monomer and alkyl cyanoacrylate oligomer.

10. The composition of claim 1, having a viscosity between about 15 to about 35 centipoise.

11. The composition of claim 1, substantially free from viscosity-modifying amounts of alkyl cyanoacrylate polymer.

12. The composition of claim 1, wherein the single container is substantially opaque to ultraviolet light.

13. A method of preparing an alkyl cyanoacrylate monomer of formula (I) 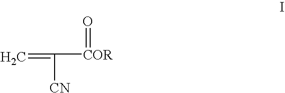
where R is alkyl of 4 to 10 carbon atoms, comprising,
(a) reacting formaldehyde with a compound of formula (1-A) 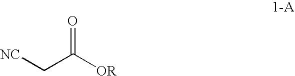
in the presence of a catalyst to provide a partial alkyl cyanoacrylate polymer;
(b) adding a first inhibitor selected from the group consisting of 4-methoxyphenol, 2,6-di-tert-butyl-4-methylphenol, SO₂ and any combinations thereof to the partial alkyl cyanoacrylate polymer;
(c) cracking the partial alkyl cyanoacrylate polymer to provide a cracked alkyl cyanoacrylate in combination with a second inhibitor selected from the group consisting of 4-methoxyphenol, 2,6-di-tert-butyl-4-methylphenol, SO₂ and any combinations thereof;
(d) distilling the cracked alkyl cyanoacrylate of (c) to provide a alkyl cyanoacrylate monomer distillate in combination with a third inhibitor selected from the group consisting of 4-methoxyphenol, 2,6-di-tert-butyl-4-methylphenol, SO₂ and any combinations thereof; and
(e) removing the third inhibitor from the alkyl cyanoacrylate monomer distillate.

14. The method of claim 13, wherein the purity of the alkyl cyanoacrylate monomer of formula (I) is from 98% to 100%.

15. A method of preparing a medical grade alkyl cyanoacrylate composition in a single container comprising,
(a) photochemically treating an alkyl cyanoacrylate monomer to produce an alkyl cyanoacrylate oligomer having a viscosity of from about 5 to about 1000 centipoise; and
(b) combining the alkyl cyanoacrylate oligomer with a plasticizer and an inhibitor to provide an alkyl cyanoacrylate oligomer plasticizer mixture.

16. The method of claim 15, wherein
the plasticizer is an acyl trialkyl citrate; and
the inhibitor is selected from the group consisting of 4-methoxyphenol, 2,6-di-tert-butyl-4-methylphenol, hydroquinone, phosphoric acid, sulfur dioxide (SO₂), and any combinations thereof.

17. The method of claim 15, further comprising,
combining an opacificant agent with the alkyl cyanoacrylate oligomer plasticizer mixture,
wherein the opacificant agent is selected from the group consisting of gold, platinum, tantalum, titanium, tungsten and barium sulfate, and any combinations.

18. The method of claim 15, wherein the single container is substantially opaque to ultraviolet light.

19. A method of providing a single container alkyl cyanoacrylate formulation comprising,
(a) providing an alkyl cyanoacrylate oligomer of a monomer of formula (I) 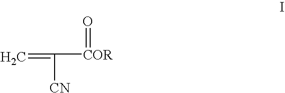
where R is alkyl of 4 to 10 carbon atoms and the monomer had a viscosity of from about 3 centipoise to about 5 centipoise prior to oligomerization; and
the alkyl cyanoacrylate oligomer has a viscosity of from about 10 centipoise to about 1000 centipoise;
(b) combining the alkyl cyanoacrylate oligomer with a plasticizer and an inhibitor to provide an alkyl cyanoacrylate oligomer plasticizer mixture; and
(c) placing the resulting alkyl cyanoacrylate oligomer plasticizer mixture in a single container, such that the resulting single container alkyl cyanoacrylate formulation is stable for more than one month at room temperature, and is adapted to polymerize in vivo.

20. The method of claim 19, wherein the plasticizer is an acyl trialkyl citrate; and wherein the inhibitor is selected from the group consisting of 4-methoxyphenol, 2,6-di-tert-butyl-4-methylphenol, hydroquinone, phosphoric acid, sulfur dioxide (SO₂), and any combinations thereof.

21. The method of claim 19, further comprising,
combining the alkyl cyanoacrylate oligomer with an opacificant agent, wherein the opacificant agent is selected from the group consisting of gold, platinum, tantalum, titanium, tungsten, an iodine compound, and barium sulfate.

22. The method of claim 18, wherein the single container is opaque to ultraviolet light.

23. A composition comprising:
(a) an alkyl cyanoacrylate oligomer;
(b) at least one inhibitor;
(c) an opacificant agent; and
(d) a plasticizer;
wherein the alkyl cyanoacrylate oligomer has been prepared from the alkyl cyanoacrylate monomer of claim 13;
wherein said composition is in a single container and is stable for more than one month, and
when said composition contacts an anionic environment it polymerizes to form an aggregate structure.

24. The composition of claim 23, wherein the alkyl cyanoacrylate oligomer is n-hexyl cyanoacrylate oligomer.

25. The composition of claim 24, wherein the n-hexyl cyanoacrylate oligomer has a viscosity of 15 to 500 centipoise.

26. The composition of claim 23, wherein the inhibitor is selected from the group consisting of 4-methoxyphenol, 2,6-di-tertbutyl-4-methylphenol, sulfur dioxide (SO₂), hydroquinone, phosphoric acid, and any combination thereof.

27. The composition of claim 23, wherein the opacificant agent is selected from the group consisting of gold, platinum, tantalum, titanium, tungsten and barium sulfate.

28. The composition of claim 27, wherein the opacificant agent is gold.

29. The compositions of claim 23, wherein the single container is substantially opaque to ultraviolet light.

30. A composition comprising:
(a) an alkyl cyanoacrylate oligomer, wherein about 30% to about 50% of the composition by weight is said alkyl cyanoacrylate oligomer, wherein the alkyl cyanoacrylate oligomer has a viscosity of from about 15 centipoise to about 500 centipoise;
(b) a plasticizer mixture, wherein 10% to 30% of said composition by weight is said plasticizer mixture; and
(c) an opacificant agent, wherein 30% to 50% of the composition by weight is said opacificant agent,
wherein said composition is in a single container and is storage stable therein at room temperature for at least about one month.

31. The composition of claim 30, wherein the plasticizer mixture consists of tributyl 2-acetylcitrate, 4-methoxyphenol, and 2,6-di-tert-butyl-4-methylphenol;
wherein the amount of 4-methoxyphenol is from about 100 to about 500 ppm, and
wherein the amount of 2,6-di-tert-butyl-4-methylphenol is from about 100 to about 500 ppm.

32. The composition of claim 30, further comprising sulfur dioxide.

33. The composition of claims 30, wherein the single container is opaque to visible light.

34. A method of preparing an embolic agent comprising,
(a) combining an alkyl cyanoacrylate oligomer with a plasticizer solution and an opacificant agent to form a mixture;
(b) sealing the mixture a single container under an inert atmosphere; and
(c) heating the single container containing the mixture to a temperature sufficient to sterilize the mixture; wherein the alkyl cyanoacrylate oligomer has a viscosity of from about 15 centipoise to about 500 centipoise; and wherein the sterilized mixture is storage stable at room temperature for at least about one month.

35. The method of claim 34, wherein the plasticizer solution is selected from the group consisting of tributyl 2-acetylcitrate, p-methoxyphenol, 2,6-di-tert-butyl-4-methylphenol, sulfur dioxide, and any combinations.

36. The method of claim 34, wherein the opacificant agent is selected from the group consisting of gold, platinum, tantalum, titanium, tungsten and barium sulfate.

37. The method of claim 36, wherein the opacificant agent is gold.

38. The method of claim 34, wherein the temperature sufficient to sterilize the pre-sterilization mixture is from about 150° C. to about 200° C.

39. The method of claim 34, wherein the single container is substantially opaque to ultraviolet light.

40. A formulation for body space remodeling, comprising;
an alkyl cyanoacrylate in an amount up to about 50 weight percent;
a plasticizer mixture in an amount up to about 30 weight percent, wherein the plasticizer mixture consists of an acyl trialkyl citrate, 4-methoxyphenol, 2,6-di-tert-butyl-4-methylphenol, sulfur dioxide, and combinations thereof; and
a opacificant agent in an amount up to 50 weight percent wherein the contrast agent is selected from the group consisting of gold, platinum, tantalum, titanium, tungsten and barium sulfate; wherein the formulation is chemically and physically stable upon storage at room temperature for at least 30 days in a single sealed container.

41. The formulation of claim 40, wherein the alkyl cyanoacrylate is n-hexyl cyanoacrylate; the acyl trialkyl citrate is tri-n-butyl O-acetylcitrate; and the opacificant agent is gold.

42. A kit for embolizing a body lumen, comprising the formulation of claim 40 and a catheter or syringe configured to introduce the formulation into a body lumen.
